(19) United States
(12) Patent Application Publication
(10) Pub. No.: US 2026/0219262 A1
(43) Pub. Date: Jul. 30, 2026

(54) KIT FOR MEASURING SYMMETRIC DIMETHYLARGININE AND METHOD FOR MEASURING SYMMETRIC DIMETHYLARGININE

(71) Applicant: FUJIFILM Corporation, Tokyo (JP)

(72) Inventors: Kazuhiro NAKAMURA, Ashigarakami-gun (JP); Tadahiro MATSUNO, Ashigarakami-gun (JP)

(73) Assignee: FUJIFILM Corporation, Tokyo (JP)

(21) Appl. No.: 19/578,210

(22) Filed: Mar. 25, 2026

Related U.S. Application Data
(63) Continuation of application No. PCT/JP2024/034070, filed on Sep. 25, 2024.

(30) Foreign Application Priority Data
Sep. 26, 2023 (JP) 2023-163241
Mar. 25, 2024 (JP) 2024-047582

(51) Int. Cl. G01N 33/532; G01N 33/543

(52) U.S. Cl. CPC G01N 33/532; G01N 33/54306; G01N 2333/4704

(57) ABSTRACT

An object of the present invention is to provide a kit for measuring SDMA and a method for measuring SDMA, each of which is capable of measuring SDMA with high sensitivity and in a simple manner. According to the present invention, there is provided a kit for measuring symmetric dimethylarginine (SDMA), the kit including (1) particles modified with a first antibody that is a monoclonal antibody having specific binding affinity for SDMA, the particles having a label, (2) a solid support, (3) a first metal thin film formed on a part of the solid support, and (4) SDMA conjugated to an immunogenic substance through a linker including a first linker, which is immobilized on the first metal thin film.

### CROSS-REFERENCE TO RELATED APPLICATIONS

This application is a Continuation of PCT International Application No. PCT/JP2024/034070 filed on Sep. 25, 2024, which claims priorities under 35 U.S.C § 119(a) to Japanese Patent Application No. 2023-163241 filed on Sep. 26, 2023 and Japanese Patent Application No. 2024-047582 filed on Mar. 25, 2024. Each of the above application(s) is hereby expressly incorporated by reference, in its entirety, into the present application.

### BACKGROUND OF THE INVENTION

### 1. Field of the Invention

The present invention relates to a kit for measuring symmetric dimethylarginine (hereinafter, also referred to as SDMA) based on an antigen-antibody reaction using a solid support, and a method for measuring symmetric dimethylarginine.

### 2. Description of the Related Art

A fluorescence detection method has been widely used as a highly sensitive and easy measurement method in biomeasurement or the like. As one of the fluorescence detection methods, it is widely practiced to bring, into contact with a sample, a substance that specifically binds to a substance to be detected that has been labeled with a fluorescent dye, irradiate the sample with excitation light having a specific wavelength, and confirm the presence of the substance to be detected by detecting fluorescence emitted at that time.

In addition, in the fluorescence detection method, to improve the sensitivity, a surface plasmon fluorescence (SPF) method using an electric field enhancement effect due to plasmon resonance has been proposed. The SPF method is a method in which a sensor chip having a metal layer provided on a predetermined region on a transparent support is used in order to generate plasmon resonance, excitation light is incident at a predetermined angle greater than or equal to a total reflection angle to an interface between the support and the metal film from a surface side of the support opposite to a metal layer forming surface, surface plasmon is generated in the metal layer by the irradiation with the excitation light, and fluorescence is enhanced due to the effect of enhancing the electric field to improve the signal-to-noise ratio (S/N ratio).

Symmetric dimethylarginine (SDMA) is known as one of the important renal markers along with urinary protein and urinary creatinine, and since it becomes elevated from an early stage of renal function decline, the demand for it as a measurement item has been increasing (J. VET. Med. 2014; 25:1676-1683. Comparison of Serum Concentrations of Symmetric Dimethylarginine and Creatinine as Kidney Function Biomarkers in Cats with Chronic Kidney Disease).

WO2010/017089A and JP2022-70879A describe that SDMA is detected by a competitive immunoassay using an anti-SDMA polyclonal antibody obtained from an antiserum, and that SDMA is detected by LC-MS and ELISA. JP2023-56523A describes an antibody that can be used in an immunoassay for detecting a symmetric dimethylarginine analyte and that binds to the analyte.

WO2018/181800A describes a measurement of a substance to be measured by performing fluorescence detection by surface plasmon excitation using a kit for measuring a target substance in a biological specimen, the kit including label particles containing a first binding substance having binding affinity for the substance to be measured in the biological specimen and a first blocking agent, and a substrate including a second binding substance having binding affinity for any of the substance to be measured or the first binding substance and a second blocking agent.

### SUMMARY OF THE INVENTION

The ELISA reagent shown in FIG. 1 of WO2010/017089A is useful as an excellent technology capable of detecting SDMA having a high correlation with a liquid chromatography-mass spectrometry (LCMS) of an SDMA measurement value, but in general, the ELISA measurement requires complicated steps and a long measurement time (about 5 hours), and is not suitable for use in point-of-care applications in which a measurement result is required in a short time on the spot. On the other hand, an anti-SDMA monoclonal antibody which is preferably used in a diagnostic reagent for detecting SDMA in a short time, in a simple manner, and with high sensitivity cannot currently be obtained as a commercially available product.

An object to be achieved by the present invention is to provide a kit for measuring SDMA and a method for measuring SDMA, each of which is capable of measuring SDMA with high sensitivity and in a simple manner.

As a result of intensive studies to achieve the above object, the present inventors have found that the above object can be achieved by measuring SDMA by bringing a solution containing particles modified with a first antibody that is a monoclonal antibody having specific binding affinity for SDMA, the particles having a label, and a biological specimen containing SDMA into contact with a first metal thin film formed on a solid support, in which the SDMA conjugated to an immunogenic substance through a linker including a first linker is immobilized. The present invention has been completed based on these findings. That is, according to an aspect of the present invention, the following invention is provided.

<1> A kit for measuring symmetric dimethylarginine (SDMA), the kit comprising: (1) particles modified with a first antibody that is a monoclonal antibody having specific binding affinity for SDMA, the particles having a label; (2) a solid support; (3) a first metal thin film formed on a part of the solid support; and (4) SDMA that is immobilized on the first metal thin film and is conjugated to an immunogenic substance through a linker including a first linker.

<2> The kit according to <1>, in which the first antibody is an anti-SDMA monoclonal antibody produced by any of a rabbit single-cell picking method, a mouse iliac lymph node method, or a rat iliac lymph node method.

<3> The kit according to <1> or <, in which the first antibody is an anti-SDMA monoclonal antibody obtained by immunizing a rabbit, a mouse, or a rat with SDMA conjugated to an immunogenic substance through a linker including a second linker.

<4> The kit according to any one of <1> to <3>, in which reactivity of the first antibody to at least one of asymmetric dimethylarginine (ADMA), methylarginine, or arginine is 25% or less of reactivity of the first antibody to SDMA.

<5> The kit according to <3> or <4>, in which the linker including the second linker is a linker having no aromatic ring.

<6> The kit according to any one of <3> to <5>, in which the SDMA conjugated to the immunogenic substance through the linker including the second linker is linked through a carboxyl group of the SDMA.

<7> The kit according to any one of <3> to <6>, in which the SDMA conjugated to the immunogenic substance through the linker including the second linker is further linked through a third linker having no aromatic ring between the second linker and the SDMA.

<8> The kit according to any one of <3> to <7>, in which an amount of SDMA introduced in the SDMA conjugated to the immunogenic substance through the linker including the second linker is 3 to 25 equivalents with respect to the immunogenic substance.

<9> The kit according to any one of <3> to <8>, in which an amount of SDMA introduced in the SDMA conjugated to the immunogenic substance through the linker including the second linker is 5 to 15 equivalents with respect to the immunogenic substance.

<10> The kit according to any one of <1> to <9>, in which the SDMA conjugated to the immunogenic substance through the linker including the first linker is linked through a carboxyl group of the SDMA.

<11> The kit according to any one of <1> to <10>, in which an amount of SDMA introduced in the SDMA conjugated to the immunogenic substance through the linker including the first linker is 3 to 25 equivalents with respect to the immunogenic substance.

<12> The kit according to any one of <1> to <11>, in which an amount of SDMA introduced in the SDMA conjugated to the immunogenic substance through the linker including the first linker is 5 to 15 equivalents with respect to the immunogenic substance.

<13> The kit according to any one of <1> to <12>, in which the first antibody having specific binding affinity for SDMA is a rabbit anti-SDMA antibody monoclonal antibody, and the kit further includes: (5) a second metal thin film formed on a part of the solid support; and (6) as a second antibody, an anti-rabbit IgG antibody that is immobilized on the second metal thin film and has no specific binding to the SDMA.

<14> The kit according to any one of <1> to <12>, in which the first antibody having specific binding affinity for SDMA is a mouse anti-SDMA antibody monoclonal antibody, and the kit further includes: (5) a second metal thin film formed on a part of the solid support; and (7) as a second antibody, an anti-mouse IgG antibody that is immobilized on the second metal thin film and has no specific binding to the SDMA.

<15> The kit according to any one of <1> to <12>, in which the first antibody having specific binding affinity for SDMA is a rat anti-SDMA antibody monoclonal antibody, and the kit further includes: (5) a second metal thin film formed on a part of the solid support; and (8) as a second antibody, an anti-rat IgG antibody that is immobilized on the second metal thin film and has no specific binding to the SDMA.

<16> A method for measuring symmetric dimethylarginine (SDMA), the method comprising: (a) a step of bringing a solution containing particles modified with a first antibody that is a monoclonal antibody having specific binding affinity for SDMA, the particles having a label, and a biological specimen containing SDMA into contact with a first metal thin film formed on a solid support, in which the SDMA conjugated to an immunogenic substance through a linker including a first linker is immobilized; and (b) a step of detecting a signal from the first metal thin film.

According to the kit and the method according to an aspect of the present invention, SDMA can be measured with high sensitivity and in a simple manner.

### BRIEF DESCRIPTION OF THE DRAWINGS

FIG. 1 shows a schematic view of a sensor chip.

FIG. 2 shows an exploded view of the sensor chip.

FIG. 3 shows calibration curves of Example 1 and Comparative Example 1.

FIG. 4 shows a result of calculating SDMA concentrations of test samples A to D from the calibration curves of Example 1 and Comparative Example 1 and plotting the SDMA concentration against the SDMA concentration by ELISA.

### DESCRIPTION OF THE PREFERRED EMBODIMENTS

Hereinafter, embodiments of the present invention will be specifically described.

In the present specification, a numerical range indicated using “to” represents a range including numerical values described before and after “to” as a minimum value and a maximum value.

### [Kit for Measuring SDMA]

A kit for measuring symmetric dimethylarginine (SDMA) according to the present invention includes (1) particles modified with a first antibody that is a monoclonal antibody having specific binding affinity for SDMA, the particles having a label, (2) a solid support, (3) a first metal thin film formed on a part of the solid support, and (4) SDMA conjugated to an immunogenic substance through a linker including a first linker, which is immobilized on the first metal thin film.

### <SDMA>

It is known that an increase in a blood concentration of SDMA is associated with cardiovascular disease and kidney disease, and in recent years, it has been used for an examination for early detection of chronic kidney disease (CKD) in veterinary medicine. SDMA is derived from post-translational modification (methylation) of a protein including an arginine residue in almost all cells, and is released into the circulating blood after protein hydrolysis or breakdown, and most of SDMA is excreted by the kidney. Therefore, it is said that the numerical value of SDMA increases as the filtration ability of the kidney decreases, and as compared with an increase in the conventional creatinine (CREA) only after the loss of approximately 75% of the renal function, SDMA increases with a loss of 40% thereof.

In addition, it is known that, unlike asymmetric dimethylarginine (ADMA), which is present in blood bound to protein, SDMA is present in blood entirely in a free, unbound state.

<First Antibody that is Monoclonal Antibody Having Specific Binding Affinity for SDMA>

The first antibody in the present invention is a monoclonal antibody having specific binding affinity for SDMA. That is, the first antibody in the present invention is an anti-SDMA monoclonal antibody in which the properties of individual antibodies with respect to SDMA, which is an analyte, are preferably uniform.

The first antibody is preferably an anti-SDMA monoclonal antibody obtained by immunizing a rabbit, a mouse, or a rat with SDMA conjugated to an immunogenic substance through a linker including a second linker described later.

SDMA is a low-molecular-weight compound, and it is difficult to use it alone as an immunogen. By binding a plurality of SDMA to the immunogenic substance through a linker and using it as a substance that induces immunity, it is possible to produce an antibody specific to SDMA. A proportion of the number of SDMA to the immunogenic substance is preferably 3 to 25 equivalents and more preferably 5 to 15 equivalents. It is considered that, since a favorable immune-inducing effect is exhibited when a plurality of SDMA are conjugated to the immunogenic substance, an antibody specific to SDMA can be produced.

### <Method for Producing Antibody>

It is preferable that the monoclonal antibody having specific binding affinity for SDMA used in the present invention is an anti-SDMA monoclonal antibody produced by any of a rabbit single-cell picking method, a mouse iliac lymph node method, or a rat iliac lymph node method, which are described below.

### (Rabbit Single-Cell Picking Method)

It is known that rabbits can produce antibodies having high specificity, sensitivity, affinity, and diversity as compared with other animal species such as mice, rats, goats, and donkeys. For example, it is also known that the antibody exhibits a strong immune response to the limited epitope portion and exhibits high stability due to the presence of an additional disulfide bond.

By using an “As One cell picking system” described in “Development of fully automated single cell isolation and analysis device” (Journal of the Society of Biotechnology, Vol. 89, No. 2, pp. 72 to 78, 2011), because cells can be placed one by one into individual wells of a microchamber having tens of thousands to several hundred thousand micro-wells, cultured, and subjected to ELISA measurement, and cells having a high ability to produce a desired antibody can be collected one by one, it is possible, for example, to pick up cells excellent in antibody-producing ability from about 200,000 monoclonalized cells. Therefore, in combination with selecting rabbits as the animal species, the likelihood of establishing clones the possibility of establishing clones that produce high-performance antibodies becomes higher than with conventional methods.

### (Method for Producing Antibody by Mouse Iliac Lymph Node Method)

The antibody production by the mouse iliac lymph node method has, first, the same effect as the rat iliac lymph node method described later, that is, the lead time from the immunization to obtaining the antibody can be reduced to about ½ as compared with 2 to 3 immunizations of the spleen method, and the amount of the immunogen used can also be reduced to ½ to ⅓ (JP4098796B).

### (Method for Producing Antibody by Rat Iliac Lymph Node Method)

The antibody production by the rat iliac lymph node method has the same effects as the mouse iliac lymph node method described above, that is, the lead time from the immunization to obtaining the antibody can be reduced, the amount of the immunogen used can be reduced, and the like (JP1994-209769A (JP-H6-209769A)).

In the present invention, since the method for producing the monoclonal antibody having specific binding affinity for SDMA can be selected from among the three different methods described above, the supply stability is excellent and the risk of depletion of the manufacturing raw material is reduced.

### (Specificity of Antibody)

With the antibody of the embodiment of the present invention, the lower the reactivity with a group of compounds having structures similar to SDMA (SDMA-like compounds), as compared with the reactivity with SDMA to be measured, the higher the specificity, and the more preferably the antibody can be used. Examples of the similar compound to be considered include asymmetric dimethylarginine (ADMA), methylarginine, and arginine, and the accuracy of the kit according to the embodiment of the present invention can be improved by selecting, from among the antibodies obtained by the above method, antibodies having high specificity, which is preferable. In the anti-SDMA antibody used in the present invention, the reactivity with at least one (preferably at least two, and more preferably all three) of asymmetric dimethylarginine (ADMA), methylarginine, or arginine is preferably 25% or less, more preferably 15% or less, still more preferably 10% or less, and particularly preferably 5% or less of the reactivity of the above-described antibody to SDMA.

Here, the reactivity is a value represented by a percentage of a proportion of the anti-SDMA antibody that has reacted when a certain amount of SDMA or an SDMA-like compound is allowed to react with the anti-SDMA antibody, and can be determined by the following method as described in Examples described later.

Specifically, it means a value calculated by the following expression by measuring fluorescence intensities B(0) and B(100) at each of (1) 0 μg/100 mL and (2) 100 μg/100 mL of the concentration of SDMA by the following measurement procedure, and is referred to as reactivity R. The evaluation is performed in a concentration range exceeding 100 μg/100 mL for the concentration of SDMA and the SDMA-like compound.

Calculation expression of reactivity R:

$$Reactivity R ( \% ) = [ 1 - \{ ( B ( 100 ) / B ( 0 ) \} ] \times 100 \qquad ( Expression 1 )$$

As the experimental conditions, the concentration of the mouse anti-SDMA antibody solution in (3) of [Measurement procedure] (1) to (9) described in Examples is adjusted such that the reactivity R obtained by the above-described (Expression 1) at the concentration of 100 μg/100 mL with respect to the concentration of 0 μg/100 mL of the SDMA concentration is in a range of 60% to 97%.

Using the reactivity R of the SDMA and the SDMA-like compound obtained as described above, a ratio of the reactivity R (SDMA) to the SDMA to the reactivity R (SDMA-like compound) to the SDMA-like compound is calculated as a reactivity ratio as an indicator of the specificity of the antibody.

Calculation expression of reactivity ratio:

$$Reactivity ratio ( \% ) = [ R ( SDM A \text{-l} ike compound ) / R ( SDMA ) ] \times 100 \qquad ( Expression 2 )$$

### <Second Linker>

As described above, it is preferable that the first antibody is an antibody obtained by immunizing a rabbit, a mouse, or a rat with the SDMA conjugated to an immunogenic substance through a linker including a second linker.

The second linker in the present invention is a linker contained in a conjugate of the immunogenic substance used as an immunogen in the creation of the antibody, and the SDMA, and is a substance that connects the immunogenic substance and the Analyte.

The linker including the second linker is preferably a linker having no aromatic ring.

As the second linker, N-(4-maleimidobutyryloxy) succinimide (GMBS) is preferable. In addition, as the linker including the second linker, a compound modified with a maleimide as the second linker to an immunogenic substance such as bovine serum albumin (BSA) or keyhole limpet hemocyanin (KLH) described in paragraph 0087 of WO2010/017089A can also be used.

It is preferable that the SDMA conjugated to the immunogenic substance through the linker including the second linker is linked through a carboxyl group of the SDMA.

### <Third Linker>

It is preferable that the SDMA conjugated to the immunogenic substance through the linker including the second linker is further linked through a third linker having no aromatic ring between the second linker and the SDMA.

The third linker is a linker having a function as an assistant of the second linker, and the third linker alone is a linker that does not function as the second linker.

As the third linker, aminoethanethiol (AET) is preferable.

### <Immunogenic Substance>

The immunogenic substance is generally a substance having a property of acting as an antigen, and in the present invention, it refers to a substance that binds to the SDMA through a linker. Preferred examples of the immunogenic substance include bovine serum albumin (BSA), horseradish peroxidase (HRP), keyhole limpet hemocyanin (KLH), and the like.

An SDMA introduction amount of the SDMA conjugated to the immunogenic substance through the linker including the second linker is preferably 3 to 25 equivalents and more preferably 5 to 15 equivalents with respect to the immunogenic substance.

<Particles Modified with First Antibody and Having Label>

In the present invention, particles modified with a first antibody that is a monoclonal antibody having specific binding affinity for SDMA, the particles having a label, are used.

The material and the form of the particle are not particularly limited, and for example, organic high molecular particles such as a polystyrene bead and an inorganic particle such as a glass bead can be used. Specific examples of the material of the particles include a homopolymer obtained by polymerizing a monomer such as styrene, methacrylic acid, glycidyl (meth)acrylate, butadiene, vinyl chloride, vinyl acetate acrylate, methyl methacrylate, ethyl methacrylate, phenyl methacrylate, or butyl methacrylate, and a copolymer obtained by polymerizing two or more monomers. The particles more preferably include a polymer selected from the group consisting of polystyrene, a polystyrene copolymer, polyacrylate, a polyacrylate copolymer, poly(meth)acrylate, and a poly(meth)acrylate copolymer. The particles may be latex in which the above-described homopolymer or copolymer is uniformly suspended. In addition, examples of the particle include another organic polymer powder, inorganic substance powder, a microorganism, a blood cell, a cell membrane fragment, a liposome, a microcapsule, and the like. The particles are preferably latex particles.

In a case of using latex particles, specific examples of the material of the latex include polystyrene, a styrene-acrylic acid copolymer, a styrene-methacrylic acid copolymer, a styrene-glycidyl (meth)acrylate copolymer, a styrene-styrene sulfonate copolymer, a methacrylic acid polymer, an acrylic acid polymer, an acrylonitrile-butadiene-styrene copolymer, a vinyl chloride-acrylic acid ester copolymer, and polyvinyl acetate acrylate. As the latex, a copolymer containing at least styrene as a monomer is preferable, and a copolymer of styrene and acrylic acid or methacrylic acid is particularly preferable. The method for preparing the latex is not particularly limited, and can be produced according to an optional polymerization method. Provided that in a case where the luminescent particles according to the embodiment of the present invention is used by labeling with an antibody, the presence of a surfactant makes it difficult to immobilize the antibody. Therefore, for production of the latex, emulsifier-free emulsion polymerization, that is, emulsion polymerization in which no emulsifier such as a surfactant is used, is preferable.

The label in the particles having the label is preferably a fluorescent substance.

As the fluorescent substance, BODIPY coloring agents having a 4,4-difluoro-4-bora-3a, 4a-diaza-s-indacene (BODIPY mother nucleus) in a mother nucleus, specifically, compounds represented by (Chemical Formula 1), or compounds having a pyrromethene skeleton capable of further high-brightness luminescence, specifically, compounds represented by (Chemical Formula 2), can be used, but the fluorescent substance is not particularly limited.

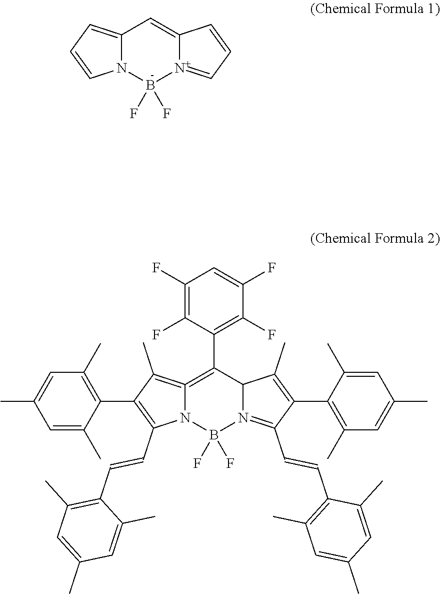

The average particle diameter of the labeled particles varies depending on the material of the particle, the concentration range of SDMA, the measuring device, and the like; however, it is preferably in a range of 0.001 to 10 μm (more preferably 0.01 to 1 μm), more preferably in a range of 30 to 500 nm, still more preferably in a range of 50 to 300 nm, particularly preferably in a range of 80 to 200 nm, and most preferably in a range of 100 to 150 nm. The average particle diameter of the labeled particles can be measured with a commercially available particle diameter distribution meter or the like. As a method for measuring the average particle diameter distribution, optical microscopy, confocal laser microscopy, electron microscopy, atomic force microscopy, static light scattering method, laser diffraction method, dynamic light scattering method, centrifugal sedimentation method, electric pulse measurement method, chromatography method, ultrasonic attenuation method, and the like are known, and apparatuses corresponding to the respective principles are commercially available. Among these measurement methods, it is preferred to measure the average particle diameter of the luminescent particles using a dynamic light scattering method from the viewpoint of the particle diameter range and ease of measurement. Examples of commercially available measuring apparatuses using dynamic light scattering include NANOTRAC UPA (Nikkiso Co., Ltd.), dynamic light-scattering particle size analyzer LB-550 (HORIBA, Ltd.), fiber-optics particle analyzer FPAR-1000 (Otsuka Electronics Co., Ltd.), and the like. In the present invention, the average particle diameter is obtained as a median diameter (d=50) measured at 25° C. under the conditions of a viscosity of 0.8872 CP and a refractive index of water of 1.330.

### <Method for Producing Labeled Particles>

A method for producing the labeled particles is not particularly limited; however, the labeled particles can be produced by mixing a fluorescence substance with particles. For example, the label particles can be produced by adding the fluorescent substance to particles such as latex particles. More specifically, the labeled particles can be produced by adding a solution containing the fluorescence substance to a dispersion liquid that contains at least one of water or a water-soluble organic solvent (tetrahydrofuran, methanol, or the like) and stirring the mixture.

In the present invention, a dispersion liquid containing the labeled particles may be prepared. The dispersion liquid can be produced by dispersing the labeled particles in a dispersion medium. Examples of the dispersion medium include water, an organic solvent, and a mixture of water and an organic solvent. An alcohol such as methanol, ethanol, or isopropanol, an ether-based solvent such as tetrahydrofuran, or the like can be used as the organic solvent.

The concentration of the solid content of the labeled particles in the dispersion liquid is not particularly limited; however, it is generally 0.1% to 20% by mass, preferably 0.5% to 10% by mass, and more preferably 1% to 5% by mass.

(Modification of Labeled Particles with First Antibody)

The method for immobilizing the first antibody on the labeled particles is described, for example, in JP2000-206115A or the protocol attached to FluoSpheres (registered trademark) polystyrene microsphere F8813 of Thermo Fisher Scientific, and any known method for preparing a reagent for an immunoagglutination reaction can be used. In addition, as a principle of immobilizing an antibody on particles, any principle of physical adsorption or a chemical bond by a covalent bond can be adopted. As a blocking agent (that is, a first blocking agent) covering a particle surface that is not coated with the antibody after immobilizing the antibody on the particle, for example, albumin (such as BSA), skim milk, casein, a soybean-derived component, a fish-derived component, polyethylene glycol, or the like, and commercially available blocking agents for an immune reaction or the like containing the substances as well as substances having the same property as that of the substances can be used. These blocking agents can be subjected to a pretreatment such as partial modification with heat, acid, alkali, or the like, as necessary. Furthermore, as the blocking agent, an antibody (globulin) which is incapable of binding to SDMA or a protein (Protein A and Protein G) which is not used in a test area can be used.

A specific method for immobilizing an antibody on particles is exemplified below. An antibody solution of which concentration is adjusted to 0.01 to 20 mg/mL is added to a liquid in which the particles are dispersed such that the concentration of the solid content of the particles becomes 0.1% to 10% by mass, and mixing is carried out. Stirring is continued for 5 minutes to 48 hours under a condition of a temperature of 4° C. to 50° C. Next, the particle and the solution are separated by centrifugation or other methods to sufficiently remove antibodies not bound to the particle contained in the solution. Then, an operation of washing the particle with a buffer solution is repeated 0 to 10 times. It is preferable that after carrying out an operation of mixing the particle with the antibody and binding the antibody to the particle, a portion of the particle surface to which the antibody is not bound is protected using a blocking agent such as a component which does not participate in the antigen-antibody reaction, preferably protein and more preferably globulin, albumin, BLOCKACE (registered trademark), skim milk, or casein.

In a case where the antigen, the antibody, or the like is immobilized on the particle, a stabilizer can be added, as necessary. The stabilizer is not particularly limited as long as it stabilizes an antigen or an antibody, such as a synthetic polymer or a natural polymer such as sucrose or polysaccharides, and a commercially available stabilizer such as Immunoassay Stabilizer (Advanced Biotechnologies Inc. (ABI)) can also be used.

The labeled particle having the first antibody is contained in the kit according to the embodiment of the present invention, and an aspect in which the labeled particle is contained in a container, for example, a cup, which is a part of the kit is preferable. In this case, the substance to be measured in the biological specimen can be bound to the first binding substance by injecting the biological specimen into a container containing the labeled particles and mixing and stirring the mixture.

### <SDMA Conjugated to Immunogenic Substance Through Linker Including First Linker, Immobilized on First Metal Thin Film>

In the present invention, the SDMA that is bound to the immunogenic substance through a linker as a crosslinking agent is immobilized on the metal thin film.

It is preferable that the SDMA conjugated to the immunogenic substance through the linker including the first linker is linked through a carboxyl group of the SDMA.

To detect the SDMA, which is an analyte, in the sample with high sensitivity, it is preferable to increase the probability that SDMA is present on the metal thin film, and it is preferable that it be a conjugate in which a plurality of SDMA are bound to the immunogenic substance. An SDMA introduction amount of the SDMA conjugated to the immunogenic substance through the linker including the first linker is preferably 3 to 25 equivalents and more preferably 5 to 15 equivalents with respect to the immunogenic substance.

As a method of immobilizing the SDMA conjugated to the immunogenic substance on the first metal thin film, any of a method using physical adsorption or a method using chemical bonding by covalent bonding can be adopted. After the SDMA conjugated to the immunogenic substance is immobilized on the first metal thin film, the surface of the solid support not coated with the SDMA conjugated to the immunogenic substance may be covered with a blocking agent. As the blocking agent, a known substance, for example, BSA, globulin, skim milk, casein, a soy-derived component, a fish-derived component, polyethylene glycol, or the like, and a commercially available blocking agent for an immunoreaction including the above-described substance or a substance having the same property as the above-described substance can be used. These blocking agents can be subjected to a pretreatment such as partial modification with heat, acid, alkali, or the like, as necessary.

### <First Linker>

The first linker is a linker contained in a conjugate of the immunogenic substance immobilized on a metal surface and the SDMA. As the first linker, N-(4-maleimidobutyryloxy) succinimide (GMBS) or m-maleimidobenzoyl-N-hydroxysuccinimide ester (MBS) is preferable.

### <Specific Example of Configuration of Kit>

Specific examples of a more preferable configuration of the kit according to the embodiment of the present invention are shown below.

### (A)

The kit in which the first antibody having specific binding affinity for SDMA is a rabbit anti-SDMA antibody monoclonal antibody, and the kit further includes: (5) a second metal thin film formed on a part of the solid support; and (6) as a second antibody, an anti-rabbit IgG antibody that is immobilized on the second metal thin film and has no specific binding to the SDMA.

### (B)

The kit in which the first antibody having specific binding affinity for SDMA is a mouse anti-SDMA antibody monoclonal antibody, and the kit further includes: (5) a second metal thin film formed on a part of the solid support; and (7) as a second antibody, an anti-mouse IgG antibody that is immobilized on the second metal thin film and has no specific binding to the SDMA.

### (C)

The kit in which the first antibody having specific binding affinity for SDMA is a rat anti-SDMA antibody monoclonal antibody, and the kit further includes: (5) a second metal thin film formed on a part of the solid support; and (8) as a second antibody, an anti-rat IgG antibody that is immobilized on the second metal thin film and has no specific binding to the SDMA.

In the aspects (A) to (C) described above, a signal caused by the particles having the label, which reacts with any of the anti-rabbit IgG antibody, the mouse IgG antibody, or the rat IgG antibody, each formed on the second metal thin film, can act as a positive control for correcting a signal caused by the particles having the label, which reacts with the SDMA conjugated to the immunogenic substance through the first linker formed on the first metal thin film.

### <Solid Support>

In the present invention, in order to achieve high-sensitive measurement, it is preferable to adopt a measurement method for carrying out surface plasmon fluorescence (SPF) detection described later. As the solid support in this case, a solid support having a metal film on the surface is preferably used. A metal constituting the metal film is not particularly limited as long as the metal can cause surface plasmon resonance. Preferably, free-electron metals such as gold, silver, copper, aluminum, or platinum can be mentioned, and gold is particularly preferable. In a case where gold is used, the detection area described later is on the gold film. The metals can be used singly or in a combination thereof. In addition, in consideration of the adhesiveness to the solid support, an intervening layer including chromium or the like may be provided between the solid support and the layer including metal. The thickness of the metal film is randomly determined; however, for example, is preferably 1 nm or more and 500 nm or less, and particularly preferably 10 nm or more and 200 nm or less. In a case where the film thickness is greater than 500 nm, the surface plasmon phenomenon of the medium cannot be sufficiently detected. In addition, in a case of providing an intervening layer which includes chromium or the like, it is preferable that the thickness of the intervening layer is 0.1 nm or more and 10 nm or less.

The formation of the metal film may be carried out by a conventional method and can be carried out, for example, by a sputtering method, a vapor deposition method, an ion plating method, an electroplating method, a non-electrolytic plating method, or the like. However, for providing a mixed layer of a material of a solid support and a metal film to improve the adhesiveness of the metal film, it is preferable to prepare the metal film by the sputtering method. In this case, the thickness of the mixed layer of the material of the solid support and the metal film is not particularly limited as long as sufficient adhesiveness can be ensured, and 10 nm or less is preferable.

The metal film is preferably disposed on the solid support. Here, “disposed on the solid support” includes a case where the metal film is disposed to be in direct contact with the solid support and a case where the metal film is disposed not in direct contact with the solid support but in contact with the solid support through other layers. The material of the solid support that can be used in the present invention is, for example, optical glass such as BK7 (borosilicate glass), which is a type of general optical glass, or synthetic resin, specifically a substance formed of a material transparent to laser light, such as polymethyl methacrylate, polyethylene terephthalate, polycarbonate, or a cycloolefin polymer can be used. Such a solid support is preferably a material that does not exhibit anisotropy with respect to polarization and has excellent processability.

As a preferred aspect of the solid support for SPF detection, a solid support in which a gold film is vapor-deposited on polymethyl methacrylate (PMMA) can be mentioned.

### <Other Elements of Kit>

The kit of the embodiment of the present invention is used for a method for measuring SDMA. In the present invention, in the case of carrying out measurement of SDMA, the kit includes a solid support and a sensor chip including a member holding labeled particles such as fluorescent particles; however, the kit may include various instruments or apparatuses used in the measurement of a substance to be measured, such as a surface plasmon excitation apparatus and a fluorescence measurement device. Furthermore, a sample containing a known amount of the substance to be measured, an instruction manual, or the like may be included as an element of the kit.

### [Method for Measuring SDMA]

A method for measuring symmetric dimethylarginine (SDMA) according to the present invention includes (a) a step of bringing a solution containing particles modified with a first antibody that is a monoclonal antibody having specific binding affinity for SDMA, the particles having a label, and a biological specimen containing SDMA into contact with a first metal thin film formed on a solid support, in which the SDMA conjugated to an immunogenic substance through a linker including a first linker is immobilized, and (b) a step of detecting a signal from the first metal thin film.

The measurement in the present invention is interpreted as the broadest concept as long as it is the measurement of SDMA, and includes quantification of SDMA and the like. A specific aspect of the measuring method is a competition method.

In the competitive method, first, a biological specimen including SDMA and anti-SDMA antibody-labeled fluorescent particles are brought into contact with the solid support on which the SDMA is immobilized. In a case where the SDMA is not present in the biological specimen, an antigen-antibody reaction occurs on the solid support by the anti-SDMA antibody-labeled fluorescent particles and the SDMA on the solid support. On the other hand, in a case where the SDMA is present in the biological specimen, an antigen-antibody reaction occurs between the SDMA in the biological specimen and the anti-SDMA antibody-labeled fluorescent particles, and the antigen-antibody reaction between the anti-SDMA antibody-labeled fluorescent particles and the SDMA on the solid support is inhibited. After the above-described reaction is completed, there are a method of removing the anti-SDMA antibody-labeled fluorescent particles that are not bound to the SDMA on the solid support and a method of not removing the particles, but a method of not removing the unreacted substance is preferably used from the viewpoint of the measurement time and the size of the device. Next, the degree of formation of the immune complex on the solid support (that is, a complex of the anti-SDMA antibody-labeled fluorescent particles and the SDMA on the solid support) is detected as a fluorescence intensity, whereby the concentration of the SDMA in the biological specimen can be measured.

The configuration of the fluorescence measurement in the competition method can adopt any one of plate reader measurement or flow measurement, and for example, the measurement can be carried out by the following method. A plurality of samples in which the SDMA concentration is different and the amount of SDMA is known are prepared in advance, and the sample and the anti-SDMA antibody-labeled fluorescent particles are mixed in advance. The mixed solution is brought into contact with a region where the SDMA is immobilized. A fluorescence signal from the region where the SDMA is immobilized is measured as a plurality of fluorescence signals at a specific time interval while the mixed solution is in contact with the complex. From the plurality of fluorescence signals, a time-dependent change (slope) of the fluorescence amount is obtained at each SDMA concentration. The time-dependent change is plotted on the Y-axis and the SDMA concentration is plotted on the X-axis, and by using an appropriate fitting method such as a least-squares method, an equation for the relationship of the SDMA concentration to the time-dependent change in the fluorescence amount is obtained. Based on the equation for the relationship acquired in this way, the amount of SDMA contained in the biological specimen can be quantified by using the result of the time-dependent change of the fluorescence amount using the biological specimen for the examination purpose.

It is preferable that the quantification of the SDMA is performed in a short time. Specifically, the quantification is preferably carried out within 10 minutes, more preferably within 8 minutes, and still more preferably within 6 minutes. This quantification time preferably includes the time required to convert the amount of SDMA which is contained in the biological sample, based on the result of the temporal change in the fluorescence amount acquired using the biological sample to be tested after the sample and the anti-SDMA antibody-labeled fluorescent particles are brought into contact with detection area where the SDMA is immobilized, by using the relational expression between the temporal change in the fluorescence amount and the SDMA concentration, which is acquired in advance using an appropriate fitting method such as the least squares method.

### (Flow Path)

In a preferred aspect of the present invention, a mixed solution obtained by mixing a biosample being likely to contain SDMA with the labeled particles having the first antibody can be applied to the solid support and developed through a flow channel. The flow path is not particularly limited as long as the path is a passage that allows the biological specimen and the label particles having the first antibody to flow down to the detection area. A preferred aspect of the flow path is a structure in which a spotting port for spotting a biological specimen solution including label particles having the first antibody, a metal film as the detection area, and a flow path present beyond the metal film are provided, and the biological specimen can pass on the metal film. Preferably, an aspiration port can be provided on a side opposite to the spotting port with respect to the metal film.

### (Surface Plasmon Fluorescence Measurement)

The method for detecting a label such as fluorescence in the present invention is not particularly limited. For example, it is preferable that fluorescence intensity is detected using a device capable of detecting fluorescence intensity, specifically, a microplate reader or a biosensor for carrying out fluorescence detection by surface plasmon excitation (SPF). Preferably, label information related to the amount of the substance to be measured can be acquired by fluorescence detection by using surface plasmon resonance.

The configuration of measurement of the fluorescence may be plate reader measurement or flow measurement. A fluorescence detection method (SPF method) by surface plasmon excitation can measure with higher sensitivity than a fluorescence detection method (epi-fluorescence method) using epi-illumination excitation, and since B/F separation is also unnecessary, the apparatus can be made smaller and the measurement time can be shortened. Therefore, the SPF method is preferably used.

As a surface plasmon fluorescence (SPF) biosensor, a sensor described in JP2008-249361A, comprising: an optical waveguide formed of a material which transmits excitation light of a predetermined wavelength; a metal film formed on one surface of the optical waveguide; a light source for generating a light beam; an optical system for passing the light beam through the optical waveguide and causing the light beam to be incident on an interface between the optical waveguide and the metal film at an incidence angle generating the surface plasmon; and fluorescence detection means for detecting fluorescence generated by being excited by an evanescent wave enhanced due to the surface plasmon can be used.

The fluorescence detection (SPF) system by surface plasmon excitation using the fluorescent particles according to the embodiment of the present invention is preferably an assay method for detecting fluorescence from the fluorescent substance depending on the amount of the SDMA immobilized on the metal film on the solid support, and for example, is a method different from a so-called latex agglutination method in which a change in optical transparency by the progress of a reaction in a solution is detected as turbidity. In the latex agglutination method, an antibody-sensitized latex in a latex reagent and an antigen in a biological specimen are bound to be agglutinated by an antibody reaction. The latex agglutination method is a method in which the agglutinate increases over time and the SDMA concentration is quantified from the change in absorbance per unit time obtained by irradiating the agglutinate with near-infrared light. In the present invention, it is possible to provide a method for measuring SDMA that is very simple and highly sensitive as compared with the latex agglutination method.

### (Standardization)

Furthermore, the method according to the embodiment of the present invention may be a method including a labeled particle-related label information acquisition step of acquiring label information related to the amount of the labeled particle; and a standardization step of standardizing label information acquired in a substance to be measured-related label information acquisition step of acquiring label information related to the amount of the SDMA, by the label information acquired in the labeled particle-related label information acquisition step.

In a step of bringing a liquid mixture containing a biological specimen and labeled particles having a first antibody having binding affinity for SDMA into contact with a solid support having a detection area (test area) and a reference area (control area) to generate the surface plasmon on the detection area and the reference area, and measuring intensity of emitted fluorescence, a step of measuring intensity of the fluorescence by the surface plasmon generated on the detection area is the substance to be measured-related label information acquisition step of acquiring label information related to the amount of SDMA, and a step of measuring intensity of the fluorescence by the surface plasmon generated on the reference area is the labeled particle-related label information acquisition step. A step of acquiring an increase rate in the unit time of the fluorescence intensity acquired in these two steps as change rate of fluorescence signal values and dividing a change rate of signal values of the detection area by a change rate of the signal value of the reference area is a standardization step.

Hereinafter, the present invention will be described in more detail with reference to examples of the present invention. The materials, amounts of use, proportions, treatment contents, treatment procedures, and the like shown in the following Examples can be appropriately modified without departing from the spirit and scope of the present invention. Accordingly, the scope of the present invention is not limited to the following specific examples.

### EXAMPLES

### [Synthesis Example of Raw Materials to be Used]

### 1. Production of SDMA-Immunogenic Substance Conjugate

### (1) Production of SDMA-AET-GMBS-BSA (Carboxyl Group Crosslinking)

SDMA-AET-GMBS-BSA (carboxyl group crosslinking) (AET: Aminoethanethiol, GMBS: N-(4-Maleimidobutyryloxy) succinimide, BSA: Bovine Serum Albumin) was synthesized according to the procedures described in Schemes 1 and II of WO2010/017089A using SDMA (manufactured by Med Chem Express Inc., Catalog No. HY-101410-50 mg) as a starting material. In Scheme II, maleimide-activated BSA was used as the maleimide-activated protein. The number of SDMA introductions per BSA molecule was produced in four types by adjusting the amount to be charged, and was determined to be 6 equivalents, 10 equivalents, 20 equivalents, and 30 equivalents from MALDI-MS measurement.

### (2) Production of SDMA-AET-MBS-HRP (Carboxyl Group Crosslinking)

SDMA-AET-MBS-HRP (carboxyl group crosslinking) (MBS: m-maleimidobenzoyl-N-hydroxysuccinimide ester, HRP: horseradish peroxidase) was synthesized according to the procedures described in Schemes 1 and II of WO2010/017089A using SDMA (manufactured by Med Chem Express Inc., Catalog No. HY-101410-50 mg) as a starting material. In Scheme II, maleimide-activated HRP was used as the maleimide-activated protein. The number of SDMA introductions per HRP molecule was determined to be 10 equivalents from MALDI-MS measurement.

### (3) Production of SDMA-MBS-BSA (α-Amino Group Crosslinking)

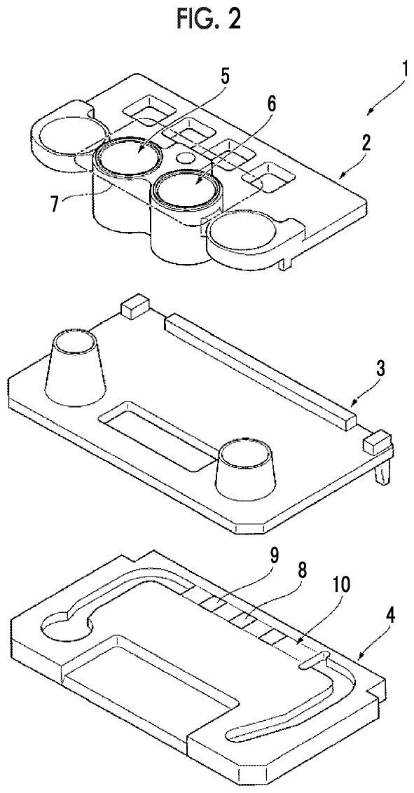

SDMA-MBS-BSA (α-amino group crosslinking) was synthesized according to the procedures described in FIG. 2 and the corresponding text of Reference 1 using SDMA (manufactured by Med Chem Express Inc., Catalog No. HY-101410-50 mg) as a starting material. In FIG. 2 of Reference 1, SDMA was used as a Hapten. The number of SDMA introductions per BSA molecule was determined to be approximately 16 equivalents from the change in molecular weight before and after the introduction.

Reference 1: Enzyme Immunoassay of Blasticidin S with High Sensitivity: A New and Convenient Method for Preparation of Immunogenic (Hapten-Protein) Conjugates (J. Biochem. 92, 585-590 (1982))

### 2. Production of Anti-SDMA Monoclonal Antibody

### (1) Production of Rabbit Anti-SDMA Monoclonal Antibody by Rabbit Single-Cell Picking Method

The three rabbits were immunized with the above-described 1. (1) SDMA-AET-GMBS-BSA (carboxyl group crosslinking, number of SDMA introductions=10 equivalents) as an immunogen, and peripheral blood mononuclear cells (PBMC) were collected 10 weeks later. Subsequently, the PBMC culture supernatant was evaluated by ELISA to select a solid positive for SDMA. Subsequently, in a single cell assay system AS ONE Cell Picking System (manufactured by AS ONE Corporation) based on Reference 2, the obtained positive cells were subjected to an immunochamber method in which a microchamber was coated with 1. (2) SDMA-AET-MBS-HRP (carboxyl group crosslinking) to select a single cell having high antibody-producing ability, and the antibody gene was acquired to produce a recombinant rabbit anti-SDMA monoclonal antibody.

Reference 2: Development of fully automated single cell isolation and analysis device (Journal of the Society of Biotechnology, Vol. 89, No. 2, pp. 72 to 78, 2011)

### (2) Production of Mouse Anti-SDMA Monoclonal Antibody by Iliac Lymph Node Method

The three mice were immunized with the above-described 1. (1) SDMA-AET-GMBS-BSA (carboxyl group crosslinking, number of SDMA introductions=10 equivalents) as an immunogen, and the mouse anti-SDMA monoclonal antibody was produced as follows.

The immunogen (SDMA-AET-GMBS-BSA (carboxyl group crosslinking, number of SDMA introductions=10 equivalents)) was injected once into the left and right tail root parts of one mouse at 100 μg per mouse, and 14 days later, the iliac lymph nodes were taken out and fused with SP2 myeloma cells using a polyethylene glycol solution. After the cell fusion, the fused cells were seeded in four 96-well culture plates. The culture supernatant after 1 week of fusion was analyzed by ELISA using a plate in which SDMA-MBS-BSA (α-amino group crosslinking) was adsorbed as an analysis antigen to determine whether or not the desired antibody was produced. The wells having an absorbance of more than 0.3 were set as positive wells.

The obtained positive wells were subjected to monoclonalization and screening by an enzyme-linked immunosorbent assay (ELISA) method to obtain a mouse anti-SDMA monoclonal antibody.

The reactivity of the obtained mouse anti-SDMA monoclonal antibody to SDMA and SDMA-like compounds (asymmetric dimethylarginine (ADMA), methylarginine, and arginine) was evaluated.

From the clinical significance, the quantification range of SDMA was set to 0 to 100 μg/100 mL, and a value calculated by the following expression by measuring fluorescence intensities B (0) and B (100) at each of (1) 0 μg/100 mL and (2) 100 μg/100 mL of the concentration of SDMA by the following measurement procedure was defined as reactivity R. The evaluation was performed in a concentration range exceeding 100 μg/100 mL for the concentration of SDMA and the SDMA-like compound.

Calculation expression of reactivity R:

$$Reactivity R ( \% ) = [ 1 - \{ ( B ( 100 ) / B ( 0 ) \} ] \times 100 \qquad ( Expression 1 )$$

As the experimental conditions, the concentration of the mouse anti-SDMA antibody solution in (3) of the following measurement procedure was adjusted such that the reactivity R obtained by the above-described (Expression 1) at the concentration of 100 μg/100 mL with respect to the concentration of 0 μg/100 mL of the SDMA concentration was in a range of 60% to 97%.

Using the reactivity R of the SDMA and the SDMA-like compound obtained as described above, a ratio of the reactivity R (SDMA) to the SDMA to the reactivity R (SDMA-like compound) to the SDMA-like compound was calculated as a reactivity ratio as an indicator of the specificity of the antibody.

Calculation expression of reactivity ratio:

$$Reactivity ratio ( \% ) = [ R ( SDM A \text{-l} ike compound ) / R ( SDMA ) ] \times 100 \qquad ( Expression 2 )$$

### [Measurement Procedure]

(1) Anti-mouse IgG antibody (Fc antibody, 50 μL of a solution of 2.5 μg/mL) was coated on a plate.
(2) The plate was washed three times with a washing agent (PBS/0.05% Tween100).
(3) The mouse anti-SDMA antibody solution was coated in an amount (50 μL of a solution of 3.3 mg/mL) within the range of reactivity described above.
(4) Solutions adjusted to concentrations of (1) 0 μg/100 mL, (2) 1 μg/100 mL, (3) 10 μg/100 mL, (4) 100 μg/100 mL, and (5) 1,000 μg/100 mL of any one of SDMA, ADMA, N-methylarginine, or arginine were coated.
(5) HRP-labeled SDMA solution (synthesized in the above-described “(2) Production of SDMA-AET-MBS-HRP (carboxyl group crosslinking)”) (50 μL of a solution of 1.1 μg/mL) was coated and allowed to react at room temperature for 30 minutes.
(6) The plate was washed three times with a washing agent (PBS/0.05% Tween100).
(7) The substrate (3,3′,5,5′-tetramethylbenzidine, manufactured by Dojindo Laboratories) was allowed to react and was incubated in the dark for 30 minutes.
(8) A reaction terminating agent (2N sulfuric acid) was added to stop the reaction.
(9) The fluorescence intensity B at a wavelength of 450 nm was measured.

The results are shown in Tables 1 to 4. The measurement was performed at an antibody concentration at which the reactivity R to SDMA was 87%, and the reactivity ratio (to SDMA) was 1.1% in ADMA, −3.4% in arginine, and 8.7% in methylarginine, all of which were less than 25%, indicating that the specificity of the present antibody to SDMA was high.

TABLE 1
SDMA

| μg/dL | B | B/B0 (%) |
|---|---|---|
| 0 | 0.878 | 100.0 |
| 0.1 | 0.861 | 98.1 |
| 1 | 0.795 | 90.5 |
| 10 | 0.579 | 65.9 |
| 100 | 0.114 | 13.0 |
| 1,000 | 0.001 | 0.1 |

R 87
Reactivity ratio (to SDMA) 100%

TABLE 2
ADMA

| μg/dL | B | B/B0 (%) |
|---|---|---|
| 0 | 0.901 | 100.0 |
| 0.1 | 0.878 | 97.4 |
| 1 | 0.864 | 95.9 |
| 10 | 0.871 | 96.7 |
| 100 | 0.892 | 99.0 |
| 1,000 | 0.887 | 98.4 |

R 1.0
Reactivity ratio (to SDMA) 1.1%

TABLE 3
Arginine

| μg/dL | B | B/B0 (%) |
|---|---|---|
| 0 | 0.883 | 100.0 |
| 0.1 | 0.872 | 98.8 |
| 1 | 0.855 | 96.8 |
| 10 | 0.875 | 99.1 |
| 100 | 0.913 | 103.4 |
| 1,000 | 0.931 | 105.4 |

R −3.4
Reactivity ratio (to SDMA) −3.9%

TABLE 4
N-methylarginine

| μg/dL | B | B/B0 (%) |
|---|---|---|
| 0 | 0.898 | 100.0 |
| 0.1 | 0.887 | 98.8 |
| 1 | 0.891 | 99.2 |
| 10 | 0.891 | 99.2 |
| 100 | 0.830 | 92.4 |
| 1,000 | 0.539 | 60.0 |

R 7.6
Reactivity ratio (to SDMA) 8.7%

### (3) Production of Rat Anti-SDMA Monoclonal Antibody by Iliac Lymph Node Method

The three rat were immunized with the above-described 1. (1) SDMA-AET-GMBS-BSA (carboxyl group crosslinking, number of SDMA introductions=10 equivalents) as an immunogen, and the rat anti-SDMA monoclonal antibody was produced as follows.

SDMA-AET-GMBS-BSA (carboxyl group crosslinking, number of SDMA introductions=10 equivalents) was used as an immunogen, and was injected into the back of the hind limb of the rat together with an immunopotentiator, and 3 weeks later, the cells of the enlarged lymph node were fused with mouse myeloma cells. The culture supernatant after 10 days was analyzed by ELISA using a plate in which SDMA-MBS-BSA (α-amino group crosslinking) was adsorbed as an analysis antigen to determine whether or not the desired antibody was produced. The wells having an absorbance of more than 0.3 were set as positive wells, and the rat anti-SDMA monoclonal antibody was extracted from the positive wells.

### (4) Preparation of Rabbit Anti-SDMA Polyclonal Antibody

A commercially available rabbit anti-SDMA polyclonal antibody (manufactured by Merck Millipore, SYM10, Catalog No. 07-412) was purchased.

### [Production of Reagent for Measuring SDMA (Sensor Chip)]

1. Production of Fluorescent Labeled Particles Modified with Anti-SDMA Antibody

### (1) Production of Latex Particles Having Average Particle Diameter of 150 nm

30 g (288 mmol) of styrene (manufactured by FUJIFILM Wako Pure Chemical Corporation) and 3 g (42 mmol) of acrylic acid (manufactured by FUJIFILM Wako Pure Chemical Corporation) were suspended in 440 mL of ultrapure water, the solution was heated to 95° C., an aqueous solution obtained by dissolving 1 g of potassium persulfate (KPS) (manufactured by FUJIFILM Wako Pure Chemical Corporation) in 10 mL of ultrapure water was added thereto, and the resulting solution was stirred at 95° C. and 250 rpm for 6 hours. Thereafter, centrifugation was performed three times at 10,000 rpm for 6 hours, thereby obtaining latex particles. Finally, the obtained latex particles were redispersed in ultrapure water. Pure water was added thereto such that the concentration of solid contents reached 1% by mass, thereby preparing a diluted solution. The average particle diameter of the latex particles was 150 nm in a case of acquiring the diameter as a median size (d=50) measured at a temperature of 25° C. using a particle size analyzer FPAR-1000 (manufactured by Otsuka Electronics Co., Ltd.).

### (2) Production of Fluorescent Latex Particles

100 mL of methanol was added to 100 mL of an aqueous dispersion liquid in which the concentration of solid contents of the latex particles with an average particle diameter of 150 nm, which had been prepared in the above-described manner, was 2% by mass, and the solution was stirred at room temperature for 10 minutes. In addition, 12 mg of a fluorescent dye 4,4-difluoro-1,3,5,7-tetraphenyl-4-bora-3a,4a,8-triazas-indacene (Chemical Formula 3) dissolved in 1 mL of N,N-dimethylformamide (DMF), 9 mL of CHCl₃, and 16 mL of ethanol, which had been separately prepared, was slowly added dropwise to the latex solution over 60 minutes. After the dropwise addition was completed, the organic solvent was distilled off under reduced pressure with an evaporator, centrifugation and redispersion in a phosphate buffered saline (PBS) aqueous solution were repeated three times, and purification was performed, thereby preparing fluorescent latex particles.

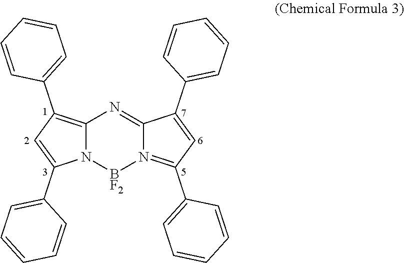

(3) Production of Fluorescent Labeled Particles Modified with Anti-SDMA Antibody

The fluorescent particles modified with the anti-SDMA antibody were prepared as follows.

39 μL of a 250 mmol/L MES (2-(N-morpholino) ethanesulfonic acid) buffer (pH 6.5) solution, 82 μL of purified water, and 29 μL of each of 1 mg/mL anti-SDMA monoclonal antibodies (antibodies produced in 2. (1) to 2. (3) and the commercially available antibody of 2. (4)) were added to 150 μL of a 2% by mass (concentration of solid contents) fluorescent latex particle aqueous solution (average particle diameter of 150 nm), and the mixture was stirred at room temperature for 15 minutes. Thereafter, 5 μL of a 10 mg/mL EDC (1-ethyl-3-(3-dimethylaminopropyl) carbodiimide hydrochloride, manufactured by FUJIFILM Wako Pure Chemical Corporation) aqueous solution was added thereto, and the solution was stirred at room temperature for 2 hours. After 8.8 μL of a 2 mol/L Glycine (manufactured by FUJIFILM Wako Pure Chemical Corporation) aqueous solution was added thereto and the solution was stirred for 15 minutes, centrifugation (15000 rpm, 4° C., 15 minutes) was performed to precipitate fluorescent latex particles. Thereafter, the supernatant was removed, 300 μL of a PBS solution (pH of 7.4) was added thereto, and the fluorescent latex particles were redispersed with an ultrasonic washing machine. After centrifugation (15000 rpm, 4° C., 15 minutes) was performed again to remove the supernatant, 300 μL of a PBS (pH of 7.4) solution containing 1% by mass of BSA was added thereto to redisperse the fluorescent latex particles, thereby preparing a 1% by mass dispersion liquid of anti-SDMA antibody-bound fluorescent latex particles (1) to (4).

2. Production of Latex Particles Modified with Anti-CRP Antibody without Fluorescent Labeling

96 μL of a 250 mmol/L MES buffer (pH of 5.6) solution and 25 mL of ultrapure water were added to 300 μL of a 2% by mass (concentration of solid contents) latex particle aqueous solution (average particle diameter of 150 nm), and 182 μL of a 5 mg/mL anti-CRP monoclonal antibody (MM50175) was added thereto. The obtained mixture was stirred at room temperature for 15 minutes. Thereafter, 9.6 μL of a 10 mg/mL EDC (N-ethyl-N′-(3-dimethylaminopropoxy) carbodiimide) aqueous solution was added thereto, and the obtained mixture was stirred at room temperature for 2 hours. 30 μL of a 2 mol/L glycine (manufactured by FUJIFILM Wako Pure Chemical Corporation) aqueous solution was added thereto, the obtained mixture was stirred for 30 minutes, and centrifugation (15000 rpm, 4° C., 15 minutes) was performed to precipitate latex particles. The supernatant was removed, 600 μL of a PBS solution (pH of 7.4) was added to the precipitate, and latex particles were redispersed with an ultrasonic washing machine. After centrifugation (15000 rpm, 4° C., 15 minutes) was performed again to remove the supernatant, 600 μL of a PBS (pH of 7.4) solution containing 1% by mass of BSA was added to the precipitate, and the latex particles were redispersed. In this manner, a 1% by mass solution of anti-CRP antibody-bound fluorescent latex particles was prepared.

### 3. Production of Dried Particles (Production of Sensor Chip Cup)

250 μL of ultrapure water, 454 μL of a 20% by mass sucrose aqueous solution, 159 μL of a 20% by mass BSA aqueous solution, 166 μL of 1% by mass anti-SDMA antibody-modified fluorescent latex particles (average particle diameter of 150 nm) produced in 1. (1) to (4), and 900 μL of a 0.8 mol/L MES buffer solution (pH=6.5) were mixed. A cup using polypropylene (Prime Polypro random PP grade, manufactured by Prime Polymer Co., Ltd.) as a base was prepared, and 17 μL of the above-described mixed solution was spotted in the cup. Thereafter, the solution was dried for 24 hours using a Super Dry Dryer (Ultra Super Dry 00 Series, manufactured by Toyo Living Co., Ltd.) until the moisture content was set to 12% or less, and the resultant was stored in an environment of 25° C. and 50% RH (relative humidity) for 15 days, thereby preparing dry particles.

### 4. Production of Sensor Chip

(1) Spotting onto Sensor Chip Solid Support

A gold film used as a test area and a gold film used as a control area next to the gold film were prepared on one surface of a solid support obtained by injection mold using polymethylmethacrylate (PMMA, ACRYPET VH-001, manufactured by Mitsubishi Rayon Co., Ltd.) as a base according to a sputtering such that the width of both the test area and the control area was set to 4 mm and the thickness thereof was set to 36 nm. A liquid (concentration: 10 μg/mL in 150 mmol NaCl) containing an SDMA-BSA conjugate produced in 1. (1) or 1. (3) was spotted on the gold vapor-deposited film in the test area of the solid support, and the substrate was incubated at 25° C. for 1 hour so that the liquid was physically adsorbed for immobilization. The liquid (concentration: 10 μg/mL in 150 mmol NaCl) containing the IgG antibody that binds to the antibody of the animal species (rabbit, mouse, or rat) of the anti-SDMA antibody applied to the fluorescent labeled latex on the sensor chip cup side was spotted onto the gold film in the control area of this solid support, and the liquid was incubated and immobilized by physical adsorption at the same time as the test area.

### (2) Washing and Blocking of Sensor Chip Solid Support

Before the sensor chip solid support prepared in this way was assembled as a sensor chip, the solid support was repeatedly washed three times using 300 μL of a washing solution (a PBS solution (pH of 7.4) containing 0.05% by mass of Tween (registered trademark) 20 (polyoxyethylene (20) sorbitan monolaurate, manufactured by FUJIFILM Wako Pure Chemical Corporation)) prepared in advance. After completion of washing, 300 μL of a PBS solution (pH of 7.4) containing 1% by mass of casein (manufactured by Thermo Scientific Inc.) was added thereto to perform blocking of a non-adsorbed portion of the antibody on the gold film, and the substrate was allowed to stand at room temperature for 1 hour. After the substrate was washed with the above-described washing solution, 300 μL of Immunoassay Stabilizer (manufactured by ABI) was added thereto as a stabilizer, the substrate was allowed to stand at room temperature for 30 minutes, and the solution was removed and the moisture was completely removed using a dryer.

### (3) Production of Sensor Chip

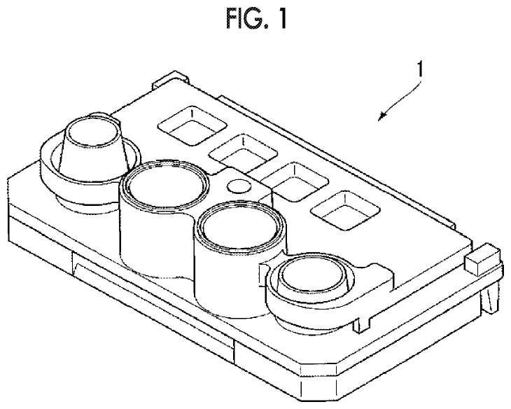

The produced sensor chip solid support and the cup were combined to produce a flow channel type sensor chip as in the configuration of the second embodiment of JP2010-190880A. FIG. 1 and FIG. 2 illustrate the schematic views thereof. FIG. 1 is a schematic view of a sensor chip 1, and FIG. 2 is an exploded view of the sensor chip 1. The sensor chip 1 is composed of an upper member (sensor chip cup) 2, an intermediate member 3, and a solid support (sensor chip solid support) 4. The upper member 2 is provided with a first container 5 and a second container 6. The first container 5 and the second container 6 are collectively referred to as a container group 7. A flow path 10 is formed in the solid support, and a detection area 8 and a reference area 9 are formed on the flow path 10.

### Examples 1 to 7 and Comparative Example 1

### 1. Sensor Chip

The sensor chips of Examples 1 to 7 and Comparative Example 1 were produced using the materials shown in Table 5 as the anti-SDMA antibody, the SDMA-BSA conjugate, and the IgG antibody. Among these, since the SDMA-AET-GMBS-BSA of Example 6 and the SDMA-MBS-BSA of Example 7 generated an insoluble substance when returned to room temperature from a frozen storage state, the sensor chip was produced through a centrifugation and filtration step.

TABLE 5

| | Fluorescent particulate anti- | | SDMA-BSA conjugate on first gold film | | | Anti-IgG |
|---|---|---|---|---|---|---|
| | SDMA antibody | | | | Amount of | antibody on |
| | | Production | | | SDMA | second gold film |
| | Antibody type | method | Linking site | Linker | introduced | Antibody type |
| Example 1 | Mouse | Iliac lymph | Carboxyl | GMBS | 10 equivalents | Anti-mouse IgG |
| | monoclonal | node method | group | | | |
| | antibody | | | | | |
| Example 2 | Rabbit | Single-cell | Carboxyl | GMBS | 10 equivalents | Anti-rabbit IgG |
| | monoclonal | picking method | group | | | |
| | antibody | | | | | |
| Example 3 | Rat monoclonal | Iliac lymph | Carboxyl | GMBS | 10 equivalents | Anti-rat IgG |
| | antibody | node method | group | | | |
| Example 4 | Rabbit | Single-cell | Carboxyl | GMBS | 6 equivalents | Anti-rabbit IgG |
| | monoclonal | picking method | group | | | |
| | antibody | | | | | |
| Example 5 | Rabbit | Single-cell | Carboxyl | GMBS | 20 equivalents | Anti-rabbit IgG |
| | monoclonal | picking method | group | | | |
| | antibody | | | | | |
| Example 6 | Rabbit | Single-cell | Carboxyl | GMBS | 30 equivalents | Anti-rabbit IgG |
| | monoclonal | picking method | group | | | |
| | antibody | | | | | |
| Example 7 | Rabbit | Single-cell | α-amino | MBS | 16 equivalents | Anti-rabbit IgG |
| | monoclonal | picking method | group | | | |
| | antibody | | | | | |
| Comparative | Rabbit | (Commercially | Carboxyl | GMBS | 10 equivalents | Anti-rabbit IgG |
| Example 1 | monoclonal | available | group | | | |
| | antibody | product) | | | | |

### 2. Production of Test Sample

The test samples (samples) A to D shown in Table 6 were produced by adding SDMA (manufactured by Med Chem Express Inc., Catalog No. HY-101410-50 mg) to the zero serum obtained by performing an activated carbon treatment on the beagle dog serum purchased from Kitayama Lab Co., Ltd., as a test substance, at each concentration. The activated carbon treatment of the diluent (zero serum: serum to which SDMA is not added) of Table 6 was performed by adding 0.6 g of activated carbon to 30 mL of beagle dog pooled serum, incubating the mixture at room temperature for 2.5 hours, and filtering the mixture through a 0.22 μm filter. For the addition of SDMA, an SDMA concentrated solution produced by dissolving 10 mg of SDMA in 10 mL of 7% BSA/PBS was used.

TABLE 6

| | Formulated | Concentration measured |
|---|---|---|
| | concentration | with control kit |
| Sample name | (μg/100 mL) | (μg/100 mL) |
| Diluent (zero serum) | 0 | 3.2 |
| A | 10 | 11.9 |
| B | 20 | 20.8 |
| C | 50 | 44.4 |
| D | 100 | 78.9 |

3. Measurement with Control Kit

In the immunometric assay, the concentration of SDMA in the test sample (zero serum, A to D) was measured by performing the measurement of the SDMA in the test sample using a commercially available ELISA kit (SDMA ELISA Kit (cat. no. K7780): manufactured by Immundiagnostik AG) according to the instructions for use. The SDMA measurement concentrations of the zero serum and the test samples A to D were 3.2 μg/100 mL, 11.9 μg/100 mL, 20.8 μg/100 mL, 44.4 μg/100 mL, and 78.9 μg/100 mL (shown together in Table 6). It took about 300 minutes to obtain this measurement result.

### 4. Immunometric Assay of SDMA

### (1) Creation of Calibration Curve

The immunometric assay was performed on the test samples A to D shown in Table 6 using the sensor chips of Examples 1 and Comparative Example 1, and a calibration curve was created.

A value (Signal 1/Signal 2) obtained by dividing the signal Signal 1 from the first metal film by the signal Signal 2 from the second metal film (both of which are the increase rate of the fluorescence intensity at a wavelength of 690 nm) was plotted against the common logarithm of the SDMA concentration of the test samples A to D in Table 6 by ELISA measurement to create a calibration curve. The values of Signal 1, Signal 2, and Signal 1/Signal 2 of Example 1 and Comparative Example 1 for each test sample are shown in Table 7. In addition, the approximate expression and the correlation coefficient in a case of linear approximation are shown in the figure. Furthermore, Log (SDMA concentration) calculated from Signal 1/Signal 2 and the approximate expression and the SDMA concentration calculated from the Log (SDMA concentration) are shown in the table.

TABLE 7

| | | SDMA | | | | | | SDMA |
|---|---|---|---|---|---|---|---|---|
| | | concentration | Log (SDMA | | | | Log (SDMA | concentration |
| | Test | ELISA | concentration) | Signal 1 | Signal 2 | Signal | concentration) | SPF (μg/100 |
| | sample | (μg/100 mL) | (ELISA) | (pA/min) | (pA/min) | 1/Signal 2 | (SPF) | mL) |
| Example 1 | D | 78.9 | 1.897 | 1,981 | 530 | 3.738 | 1.898 | 79.0 |
| | C | 44.4 | 1.647 | 2,350 | 540 | 4.350 | 1.652 | 44.8 |
| | B | 20.8 | 1.318 | 2,566 | 493 | 5.210 | 1.306 | 20.2 |
| | A | 11.9 | 1.077 | 2,710 | 470 | 5.762 | 1.084 | 12.1 |
| Comparative | D | 78.9 | 1.897 | 2,782 | 3,783 | 0.736 | 2.587 | 368.7 |
| Example 1 | C | 44.4 | 1.647 | 2,834 | 3,735 | 0.759 | 0.329 | 2.1 |
| | B | 20.8 | 1.318 | 2,803 | 3,781 | 0.741 | 2.031 | 107.4 |
| | A | 11.9 | 1.077 | 2,850 | 3,789 | 0.752 | 0.977 | 9.5 |

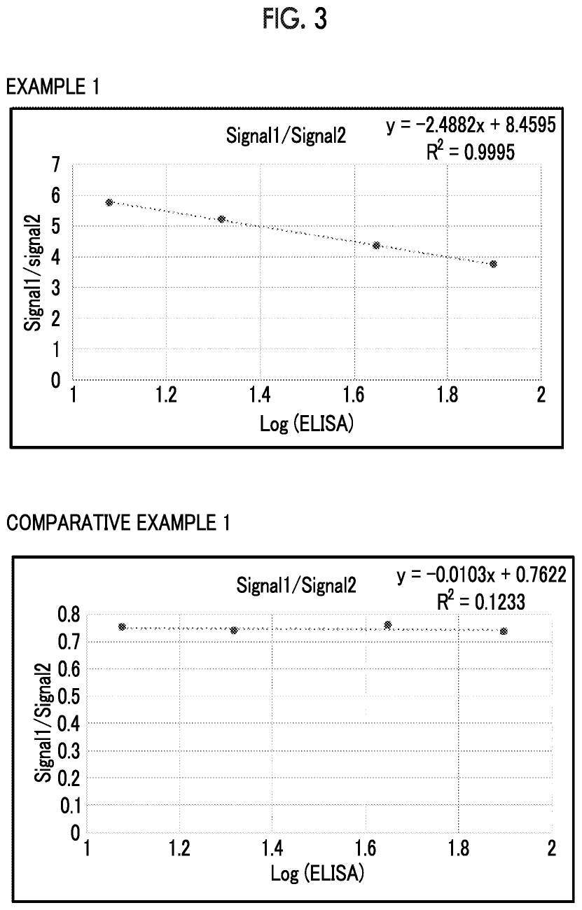

FIG. 3 shows the calibration curves of Examples 1 and Comparative Example 1. The calibration curve of Example 1 had a correlation coefficient R=0.9997 of the linear approximation expression, indicating a very good correlation. The calibration curve of Comparative Example 1 had a correlation coefficient R=0.3507, and was a calibration curve that could not be used because there was no correlation.

### (2) Conversion to SDMA Concentration

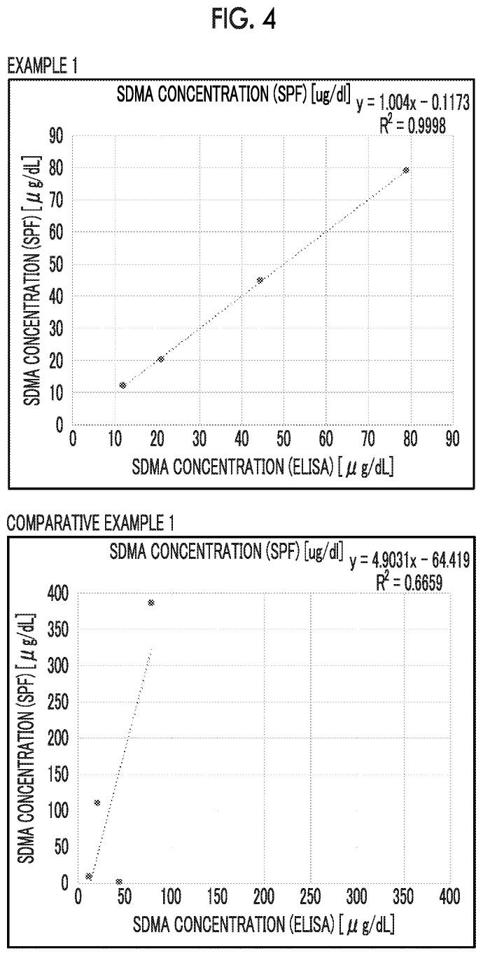

From the calibration curves of Examples 1 and Comparative Example 1, the SDMA concentrations of the test samples A to D were calculated and plotted against the SDMA concentration by ELISA (FIG. 4). In Example 1, the linear approximation between the ELISA and the SPF gave the following parameters: slope=1.004, Y-intercept=−0.12, and a correlation coefficient R=0.9999, indicating that the results were very good agreement with the ELISA results. On the other hand, in Comparative Example 1, as a result of insufficient sensitivity in the measurement range, the calibration curve showed poor correlation, so that the converted values of the SPF measurement results had very poor correlation with the ELISA results (slope of a linear approximation=4.903, y-intercept=−64.4, and correlation coefficient R=0.8160). Therefore, it was not possible to use as a quantitative measurement method.

In addition, while the measurement of the commercially available ELISA required 15 steps of operation and about 300 minutes, the measurement of Example 1 of the present invention was completed in 12 minutes by setting the sensor chip and the measurement sample in a commercially available fluorescent immunoassay analyzer FUJI DRI-CHEM IMMUNO AU10V (manufactured by FUJIFILM Corporation) and pressing a start button, and the operation was simple.

### EXPLANATION OF REFERENCES

1: sensor chip
2: upper member
3: intermediate member
4: solid support
5: first container
6: second container
7: container group
8: detection area
9: reference area
10: flow path

What is claimed is:

1. A kit for measuring symmetric dimethylarginine (SDMA), the kit comprising:
(1) particles modified with a first antibody that is a monoclonal antibody having specific binding affinity for SDMA, the particles having a label;
(2) a solid support;
(3) a first metal thin film formed on a part of the solid support; and
(4) SDMA that is immobilized on the first metal thin film and is conjugated to an immunogenic substance through a linker including a first linker.

2. The kit according to claim 1,
wherein the first antibody is an anti-SDMA monoclonal antibody produced by any of a rabbit single-cell picking method, a mouse iliac lymph node method, or a rat iliac lymph node method.

3. The kit according to claim 1,
wherein the first antibody is an anti-SDMA monoclonal antibody obtained by immunizing a rabbit, a mouse, or a rat with SDMA conjugated to an immunogenic substance through a linker including a second linker.

4. The kit according to claim 1,
wherein reactivity of the first antibody to at least one of asymmetric dimethylarginine (ADMA), methylarginine, or arginine is 25% or less of reactivity of the first antibody to SDMA.

5. The kit according to claim 3,
wherein the linker including the second linker is a linker having no aromatic ring.

6. The kit according to claim 3,
wherein the SDMA conjugated to the immunogenic substance through the linker including the second linker is linked through a carboxyl group of the SDMA.

7. The kit according to claim 3,
wherein the SDMA conjugated to the immunogenic substance through the linker including the second linker is further linked through a third linker having no aromatic ring between the second linker and the SDMA.

8. The kit according to claim 3,
wherein an amount of SDMA introduced in the SDMA conjugated to the immunogenic substance through the linker including the second linker is 3 to 25 equivalents with respect to the immunogenic substance.

9. The kit according to claim 3,
wherein an amount of SDMA introduced in the SDMA conjugated to the immunogenic substance through the linker including the second linker is 5 to 15 equivalents with respect to the immunogenic substance.

10. The kit according to claim 1,
wherein the SDMA conjugated to the immunogenic substance through the linker including the first linker is linked through a carboxyl group of the SDMA.

11. The kit according to claim 1,
wherein an amount of SDMA introduced in the SDMA conjugated to the immunogenic substance through the linker including the first linker is 3 to 25 equivalents with respect to the immunogenic substance.

12. The kit according to claim 1,
wherein an amount of SDMA introduced in the SDMA conjugated to the immunogenic substance through the linker including the first linker is 5 to 15 equivalents with respect to the immunogenic substance.

13. The kit according to claim 1,
wherein the first antibody having specific binding affinity for SDMA is a rabbit anti-SDMA antibody monoclonal antibody, and
the kit further includes:
(5) a second metal thin film formed on a part of the solid support; and
(6) as a second antibody, an anti-rabbit IgG antibody that is immobilized on the second metal thin film and has no specific binding to the SDMA.

14. The kit according to claim 1,
wherein the first antibody having specific binding affinity for SDMA is a mouse anti-SDMA antibody monoclonal antibody, and
the kit further includes:
(5) a second metal thin film formed on a part of the solid support; and
(7) as a second antibody, an anti-mouse IgG antibody that is immobilized on the second metal thin film and has no specific binding to the SDMA.

15. The kit according to claim 1,
wherein the first antibody having specific binding affinity for SDMA is a rat anti-SDMA antibody monoclonal antibody, and
the kit further includes:
(5) a second metal thin film formed on a part of the solid support; and
(8) as a second antibody, an anti-rat IgG antibody that is immobilized on the second metal thin film and has no specific binding to the SDMA.

16. A method for measuring symmetric dimethylarginine (SDMA), the method comprising:
(a) a step of bringing a solution containing particles modified with a first antibody that is a monoclonal antibody having specific binding affinity for SDMA, the particles having a label, and a biological specimen containing SDMA into contact with a first metal thin film formed on a solid support, in which the SDMA conjugated to an immunogenic substance through a linker including a first linker is immobilized; and
(b) a step of detecting a signal from the first metal thin film.
